(19) United States
(12) Patent Application Publication
(10) Pub. No.: US 2008/0305096 A1
(43) Pub. Date: Dec. 11, 2008

(54) METHOD AND COMPOSITION FOR PROVIDING CONTROLLED DELIVERY OF BIOLOGICALLY ACTIVE SUBSTANCES

(75) Inventors: Peter J. E. Verdegem, Draper, UT (US); Stewart F. Hughes, Orem, UT (US); Curtis A. Hedges, Highland, UT (US); G. Paulo Bangerter, American Fork, UT (US)

Correspondence Address: KIRTON AND MCCONKIE, 60 EAST SOUTH TEMPLE,, SUITE 1800, SALT LAKE CITY, UT, 84111, US

(73) Assignee: UNICITY INTERNATIONAL, INC., Orem, UT (US)

(21) Appl. No.: 11/759,410

(22) Filed: Jun. 7, 2007

(51) Int. Cl. A61K 47/06; A61K 31/315; A61K 31/34; A61K 31/355; A61K 33/04; A61K 33/32; A61K 36/45; A61K 47/26; A61P 3/04; A61K 36/73; A61K 36/752; A61K 36/889; A61K 36/8962; A61K 38/43; A61K 38/44

(52) U.S. Cl. 424/94.4; 424/639; 424/643; 424/650; 424/702; 424/727; 424/735; 424/736; 424/750; 424/754; 424/755; 424/765; 424/777; 424/94.1; 514/458; 514/474; 514/772; 514/777; 514/789

(57) ABSTRACT

A method of providing controlled release of a biologically active substance within a subject's digestive system. The biologically active substance is administered concurrently with one or more soluble fibers in an oral dosage unit. The soluble fibers interact with the biologically active substance within the subject's digestive system to moderate and control the release of the biologically active substances in the subject's bloodstream. This provides more constant blood concentrations of the biologically active substances. The amount of soluble fibers in the oral dosage unit is greater than 40% by weight, and in some cases greater than 50% by weight of the oral dosage unit. The oral dosage unit typically contains from about 1 to 15g of soluble fiber, and in some cases from about 3 to 5g of soluble fiber. The biologically active substance may contain phytonutrients that promote the subject's cardiovascular system, immune system, or weight management.

### BACKGROUND OF THE INVENTION

The present invention relates in general to orally administrable compositions and methods that provide controlled delivery of biologically active substances.

Most orally administered biologically active substances must pass from the digestive system into a subject's bloodstream to be effective. Once in the bloodstream, the biologically active substances circulate to the subject's cells, organs, and tissues to provide biological activity. The concentration of these substances in blood typically varies over time. Usually there is a large increase in concentration shortly after orally administering the substance, followed by a steady decrease in concentration as the substance is metabolized. Often, biologically active substances are most effective when their blood concentration is maintained at a more constant concentration or within desired concentration ranges.

It would be an improvement in the art to provide orally administrable compositions and methods that provide controlled release of biologically active substances into a subject's bloodstream. It would be a further improvement to provide orally administrable compositions and methods that maintain a substantially constant blood concentration of biologically active substances or a concentration within desired concentration ranges. Such methods and compositions are provided herein.

### BRIEF SUMMARY OF THE INVENTION

The present invention is based in part upon the discovery that soluble fibers, alone or in combination with insoluble fibers, when administered concurrently with biologically active substances, moderates and controls the release of the biologically active substances in the subject's bloodstream. This provides more constant blood concentrations of the biologically active substances.

Dietary fibers are the indigestible portion of plant foods that move food through the digestive system, absorbing water. On average, North Americans consume less than 50% of the dietary fiber levels required for good health. Diets rich in processed foods, which often contain low dietary fiber content and high carbohydrate and fat content, are believed by experts to contribute to the obesity crisis seen in many first-world western countries. Dietary fibers include soluble and insoluble fibers. The American Association of Cereal Chemists defined soluble fiber this way: “the edible parts of plants or similar carbohydrates resistant to digestion and absorption in the human small intestine with complete or partial fermentation in the large intestine. ”

Due to the growing scientific evidence for physiological benefits of increased fiber intake, regulatory agencies such as the United States Food and Drug Administration (FDA) have given approvals to food products making health claims for soluble (fermentable) fiber. In this case, the claim may state that soluble fiber (primarily beta-glucans) from food such as whole oat products, as part of a diet low in saturated fat and cholesterol may reduce the risk of coronary heart disease. The eligible sources of soluble fiber allowed for this health claim include: psyllium seed husk (7 grams per day); beta-glucan from oat bran, whole oats, oatrim or rolled oats (3 grams per day); and beta-glucan from whole grain or dry-milled barley (3 grams per day).

In clinical trials to date, these fiber sources were shown to significantly reduce blood cholesterol levels and so are important to cardiovascular health. Consistent consumption of soluble fiber may reduce risk of some of the world's most prevalent diseases—obesity, diabetes, high blood cholesterol, cardiovascular disease, and numerous gastrointestinal disorders.

One embodiment within the scope of the present invention includes a method of providing controlled release of a biologically active substance within a subject's digestive system. The subject obtains an oral dosage unit comprising the biologically active substance combined with one or more soluble fibers. The oral dosage unit is administered to the subject. The one or more soluble fibers interact with the biologically active substance within the subject's digestive system to provide controlled release of the biologically active substance. In one embodiment, the oral dosage unit is optionally administered to the subject substantially simultaneously with a meal.

The amount of soluble fibers in the oral dosage unit is preferably greater than 40% by weight. In some embodiments, the amount of soluble fibers is between 40% by weight and 95% by weight of the oral dosage unit. In other embodiments, the amount of soluble fibers is between 45% by weight and 90% by weight of the oral dosage unit. In yet other embodiments, the amount of soluble fibers is between 45% by weight and 75% by weight of the oral dosage unit. In still other embodiments, the amount of soluble fibers is between 45% by weight and 60% by weight of the oral dosage unit. In other embodiments, the amount of soluble fibers is between 45% by weight and 55% by weight of the oral dosage unit. In terms of the actual quantity of soluble fibers, the oral dosage unit contains at least 1 g of the one or more soluble fibers. Embodiments of the oral dosage unit may contain from about 1 to 15 g of soluble fiber; other embodiments of the oral dosage unit may contain from about 2 to 10 g of soluble fiber; and still other embodiments of the oral dosage unit may contain from about 3 to 5 g of soluble fiber.

The one or more soluble fibers may include a mixture of soluble fibers. Examples of common soluble fibers that may be contained in the oral dosage unit include, but are not limited to, guar gum, gum Arabic, locust bean gum, pectin, oat fiber, beta glucan, psyllium, gum acacia, xanthane gum, innuline, fructo-oligosaccharides (FOS), carrageenan, and mixtures thereof. The oral dosage unit may optionally contain one or more insoluble dietary fibers.

The oral dosage unit may contain an orally-ingestible pharmacologically-acceptable mineral salt capable of dissolution in the digestive system to release a gas. Examples of the mineral salts include, but are not limited to, a mineral carbonate compound, a mineral hydrogen carbonate compound, a mineral dihydrogen carbonate compound, or mixtures thereof. Examples of the minerals include, but are not limited to, alkali and alkaline earth metals, such as sodium, potassium, calcium, magnesium. Other biologically acceptable minerals may be used, including transition metals.

In some embodiments, the biologically active substance comprises nutritional vitamins, minerals, and phytonutrients. In other embodiments, the nutritional vitamins, minerals and phytonutrients are derived from fruits and vegetables. The vitamins and phytonutrients may include antioxidants. The antioxidants may include, but are not limited to, antioxidant plant enzymes and antioxidant vegetable concentrates.

In some embodiments, the biologically active substance may be selected to support the subject's immune system. Such substances may provide the following metabolic functions selected from, but not limited to, upregulate immune response, increase white blood cell count, and activate B-, T-, or NK-cells. In other embodiments, the biologically active substance may be selected to support the subject's cardiovascular system. Such cardiovascular functions may include, but not limited to, reducing cholesterol, reducing blood pressure, reducing arteriosclerotic plaque, helping to lower LDL cholesterol and/or increase HDL cholesterol, and helping to improve cardiac output and/or increase the pumping strength of the heart. In still other embodiments, the biologically active substance may be selected to support the subject's weight management control. Such weight management functions may include, but not limited to, decrease appetite, increase resting metabolic rate, reduce fat storing, reduce carbohydrate absorption, and reduce glycemic index.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment, but may refer to every embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

The present invention includes a method of providing controlled release of a biologically active substance within a subject's digestive system. The biologically active substance is administered concurrently with one or more soluble fibers in an oral dosage unit. The soluble fiber interacts with the biologically active substance within the subject's digestive system to moderate and control the release of the biologically active substances in the subject's bloodstream. This provides more constant blood concentrations of the biologically active substances.

As used herein, the term “controlled release” refers to the gradual release of the biologically active substance over a prolonged period of time. The term controlled release may include other terms, such as timed release, prolonged release, sustained release, or delayed release. Controlled release can be described as producing a blood concentration curve of the substance that is broader than the curve produced without soluble fiber, but with substantially equal area under the curve.

As used herein, the term “biologically active substance” refers to any substance or substances comprising an active therapeutic substance, metabolite, medicament, vitamin, or mineral, any substance used for treatment, prevention, diagnosis, cure or mitigation of disease or illness, any substance which affects anatomical structure or physiological function, or any substance which alters the impact of external influences on an animal, or metabolite thereof, and as used herein, encompasses the terms “active substance,” “therapeutic substance,” “agent,” “active agent,” “active therapeutic agent,” “medication,” “medicine,” “medicant,” and other such similar terms. In some embodiments within the scope of the invention, the biologically active substance includes nutritional vitamins, minerals, and phytonutrients.

The term phytonutrients include plant-derived compounds that have biological activity in the body. Phytonutrients naturally occur in vegetables and fruit. In broad terms, they can be said to be any biologically useful chemical or nutrient derived from a plant source. However, in common usage, they have a more limited definition. They are usually used to refer to compounds found in plants that are not required for normal functioning of the body but that nonetheless have a beneficial effect on health or an active role in the amelioration of disease.

Some phytonutrients found in fruits and vegetables have previously been classified as vitamins: flavonoids were known as vitamin P, cabbage factors (glucosinolates and indoles) were called vitamin U, and ubiquinone was vitamin Q. Tocopherol remained on the list as vitamin E. The “vitamin” designation was dropped for the other nutrients because specific deficiency symptoms could not be established. Today scientists and researchers may group phytonutrients into classes on the basis of similar protective functions as well as individual physical and chemical characteristics of the molecules. Some examples of different classes of phytonutrients include, but are not limited to, terpenes, carotenoids, limonoids, phytosterols, phenols, flavonoids, anthocyanidins, catechins and gallic acids, isoflavones, thiols, glucosinolates, allylic sulfides, indoles, isoprenoids, tocotrienols and tocopherols, lipoic acid, ubiquinone, and phytoestrogens.

As used herein, the term “digestive system” means the series of connected organs whose purpose is to break down, or digest, the food eaten by the subject. Food is made up of large, complex molecules, which the digestive system breaks down into smaller, simple molecules that can be absorbed into the bloodstream. The simple molecules travel through the bloodstream to all of the body's cells, which use them for growth, repair and energy. The digestive system specifically includes the stomach, large and small intestines.

In one embodiment within the scope of the invention, an oral dosage unit is obtained that comprises the biologically active substance combined with one or more soluble fibers. The amount of the one or more soluble fibers in the oral dosage unit is greater than 40% by weight, and more preferably from about 40% by weight and 95% by weight of the oral dosage unit. In other embodiments, the amount of soluble fibers is between 45% by weight and 90% by weight of the oral dosage unit. In yet other embodiments, the amount of soluble fibers is between 45% by weight and 75% by weight of the oral dosage unit. In still other embodiments, the amount of soluble fibers is between 45% by weight and 60% by weight of the oral dosage unit. In other embodiments, the amount of soluble fibers is between 45% by weight and 55% by weight of the oral dosage unit. The oral dosage unit is orally administered to the subject, wherein the one or more soluble fibers interact with the biologically active substance to provide controlled release of the biologically active substance within the subject's digestive system. The actual amount of soluble fibers included in the oral dosage unit may be varied depending upon the biologically active substance. For example, a potent biologically active substance may be combined with a larger amount of soluble fibers to provide greater controlled release of the biologically active substance. The oral dosage unit, and the ingredients and components thereof, are biologically acceptable. As used herein, the term “biologically acceptable” refers to being safe for human consumption.

The one or more soluble fibers may be in the form of a mixture of soluble fibers. The soluble fibers may include, but are not limited to, guar gum, gum Arabic, locust bean gum, pectin, oat fiber, beta glucan, psyllium, gum acacia, xanthane gum, innuline, fructo-oligosaccharides (FOS), carrageenan, and mixtures thereof. The oral dosage unit preferably includes at least 1 g of the soluble fiber. In one embodiment, the oral dosage unit includes from about 1 to 15 g of the soluble fiber. In another embodiment, the oral dosage unit comprises from about 2 to 10 g of the soluble fiber. In yet another embodiment, the oral dosage unit comprises from about 3 to 5 g of the soluble fiber.

In one embodiment, the oral dosage unit includes a mixture of the following soluble fibers:

TABLE 1

| Ingredient | weight percent |
|---|---|
| Guar gum | 37.5 |
| Gum Arabic | 19.7 |
| Locust bean gum | 18.3 |
| Citrus pectin | 12.8 |
| Oat fiber | 10.5 |
| Beta glucan (15% Beta glucan) | 1.2 |

The oral dosage unit may include one or more insoluble fibers. When processing natural sources of dietary fiber, it may be difficult to obtain pure soluble fiber. Measurable amounts of insoluble fiber may be present. In one embodiment within the scope of the invention, of the total dietary fiber present in the oral dosage unit, 75% to 100% by weight, may be soluble fiber. In another embodiment, the total dietary fiber content may contain from 85% to 90% by weight soluble fiber.

Useful results are observed when the oral dosage unit contains an orally-ingestible pharmacologically-acceptable mineral salt capable of dissolution in the digestive system to release a gas. The mineral salt may include, but is not limited to, a mineral carbonate compound, a mineral hydrogen carbonate compound, a mineral dihydrogen carbonate compound, or mixtures thereof. Calcium carbonate (CaCO₃) is an example of a useful mineral carbonate compound. The gas releasing mineral salt may be present in the oral dosage unit in an amount ranging from about 2% to 10% by weight, relative to the amount of soluble fiber present in the oral dosage unit. Some embodiments of the oral dosage unit contain a gas releasing mineral salt present in am amount of about 5% by weight, relative to the amount of soluble fiber present in the oral dosage unit. While not wishing to be bound by theory, the release of gas, such as carbon dioxide, in the digestive system may enhance the controlled release of biologically active substances.

The oral dosage unit may contain chromium to enhance the controlled release of biologically active substances. Chromium may be used in various forms including, but not limited to, chromium chloride, chromium sulfate, chromium enriched yeast, chromium polinicotinate, chromium picolinate, and mixtures thereof.

In one embodiment of the method within the scope of the present invention, the oral dosage unit is administered to the subject substantially simultaneously with a meal. As used herein, the term “substantially simultaneously with a meal” means during, at the beginning, or at the end of the subject's meal. The term “meal” refers any of the occasions for eating food that occur by custom or habit at more or less fixed times, including, but not limited to, breakfast, lunch, and dinner. The term substantially simultaneously with a meal is intended to exclude and distinguish administering the oral dosage unit to the subject prior to a meal or prior to the subject eating. The term “substantially simultaneously with a meal” will typically mean during the meal or less than five minute before the meal or less than five minutes after the meal.

The biologically active substances may be selected to provide a desired biological activity. For example, the biologically active substance may be selected to support a subject's immune system, support the subject's cardiovascular system, support the subject's weight management control, or support the subject's general health and wellbeing.

Biologically active substances that support the immune system may provide the following metabolic functions selected from, but not limited to, upregulate immune response, increase white blood cell count, and activate B-, T-, or NK-cells. Biologically active substances that support weight management control may provide the following metabolic functions selected from, but not limited to, decrease appetite, increase resting metabolic rate, reduce fat storing, reduce carbohydrate absorption, and reduce glycemic index. Biologically active substances that support the cardiovascular system may provide the following metabolic functions selected from, but not limited to, reduce cholesterol, reduce blood pressure, reduce arteriosclerotic plaque, help lower LDL cholesterol and/or increase HDL cholesterol, and help improve cardiac output and/or increase the pumping strength of the heart.

The biologically active substance may be obtained from various sources. In one embodiment, the biologically active substance may include nutritional vitamins, minerals and phytonutrients derived from fruits and vegetables.

Fruit and vegetable phytonutrients may be selected from, but are not limited to, acai, alfalfa, apple, artichoke, apricot, asparagus, avocado, barley grass, bilberry, beans, bittermelon, beet, blackberry, broccoli, black current, Brussels sprouts, blueberry, cabbage, cantaloupe, cassava, carrot, cherry, cauliflower, coconut, celery, coriander, cranberry, chlorella, gauvas, corn, grape, cucumber, garlic, grapefruit, horseradish, hops, kale, kava, kamut, kiwi, lima beans, lemon, oat grass, mangos, olive, orange, parsley, papaya, peach, peach, peas, pear, pepper, pineapple, potato, plum, pumpkin, pomegranate, rice, raspberries, spinach, strawberry, spirulina, tangerines, squash, tomato, sweet potatoes, wheat germ, wheat grass, white kidney beans, and mixtures thereof.

In one embodiment, the vitamins and phytonutrients are antioxidants. The antioxidants may be selected from, but not limited to, lycopene, anthocyanosides, alfalfa chlorophyll complex beta-carotene, alpha-carotene, lutein, zeaxanthin, canthaxanthin, astaxanthin, tocopherol, epigallocatechin gallate (EGCG), acetylcysteine, alpha lipoic acid, beta carotene, bilberry, burdock, carnosine, catalase, conjugated linoleic acid (CLA), CoEnzyme Q10, cryptoxanthin, curcumin, daidzein, dehydroepiandrosterone (DHEA), dimethylaminoethanol (DMAE), garlic, genistein, germanium, Ginkgo biloba, glutamine, glutathione, grape seed extract, green tea, lutein, lycopene, manganese, melatonin, methionine, para-aminobenzoic acid (PABA), pycnogenol, quercetin, resveratrol, selenium, superoxide dismutase, taurine, vitamin C (ascorbic acid), vitamin E, zeaxanthin, zinc, and mixtures thereof.

In another embodiment, the phytonutrients comprise antioxidant plant enzymes. The antioxidant plant enzymes may be selected from, but not limited to, catalase, glucose oxidase, peroxidase, superoxide dismutase, glutathione peroxidase, and mixtures thereof. In one embodiment, the nutritional vitamins and phytonutrients comprise antioxidant vegetable concentrates.

In one embodiment, the biologically active substance may include substances that support the subject's immune system. Substances that support the immune system may be selected from, but not limited to, alfalfa leaf, alpha lipoic acid, allium cepa, aloe vera, antioxidant plant enzymes, apricot extract, nectarine extract, arabinogalactan, Arnica montana, arsenicum album, bee pollen, benzenum, belladonna, beta carotene, biotin, Piper nigrum L (black pepper) extract, Piper longum L (long pepper) extract, bladderwrack (kelp) extract, boron, colostrum, burdock root, cadmium sulphuricum, calcium ascorbate, calcium citrate, capsicum, carotenoids, carrot, cat's claw, cayenne, chlorum, choline bitartrate, polynicotinate, citrus bioflavonoids (lemon), cruciferous vegetable concentrate, cupric oxide (copper), cuprum metallicum, copper glycinate, dandelion root, Drosera rotundifolia, echinacea, elderberry, ferrum phosphoricum, folic acid (folate), fructose, garlic, ginger root, golden seal root, grape seed extract, grape wine concentrate, green tea extract (leaf), guarana extract, xanthan gum, hops strobile, inositol, iron rice chelate, lactoferrin, lutein, lycopene, magnesium, manganese rice chelate, marigold flower extract, milk thistle herb, mixed berry anthocyanosides, molybdenum, Morinda citrifolia extract, mulberry, n-acetyl cysteine, nettle leaf, niacin/niacinamide, nux vomica, oxo vanadium bis glycinato, pantothenic acid, para amino benzoic acid (PABA), perilla seed extract, pine bark extract, plumbum metallicum, pomegranate extract, potassium, potassium iodide (iodine), prune extract (fruit), quercetin, red clover blossom, red wine extract, rhubarb root, rose hips, rutin, Schisandra chinensis fruit extract, scullcap herb, selenium, sheep sorrel herb, slippery elm bark, soy, tabacum, turmeric, valerian root, vitamin A palmitate, Vitamin B1/B2, Vitamin B6/B12, Vitamin C (ascorbic acid), Vitamin D (2&3), Vitamin E (d-alpha-tocopherol), Vitamin K (phytonadione), watercress leaf, CoEnzyme Q10, glutamine, hydroxymethylbutyrate (HMB), L-arginine, lentinan, red yeast, S-adenosyl- L-methionine (SAMe), sangre de grado (dragon's blood), whey protein, medium chain triglycerides (MCT), St. John's Wort, boxwood, dehydroepiandrosterone (DHEA), riboflavin (vitamin B2), Ufia de Gato extract, zinc, and mixtures thereof.

In another embodiment, the biologically active substance may include substances that support the subject's cardiovascular system. Substances that support the cardiovascular system may be selected from, but not limited to, acerola, apricot extract, nectarine extract, B-Vitamins (1, 2, 3, 6, 12), beta carotene, biotin, black current seed oil, gamma-linolenic acid (GLA), borage oil, calcium phosphate, carnosine, carnithine, carotenoids, choline bitartrate, chondroitin sulfate, coconut oil, cognis phytosterols, copper (cupric oxide), CoEnzyme Q10, D-ribose, evening primrose seed oil, fish oil, eicosapentaenoic acid (EPA), docosahexaenoic acid (DHA), flax seed oil, folic acid (folate), garlic, grape seed extract, grape juice extract, grape skin extract, green tea extract, glutathione (GSH), hawthorne berry extract, hesperidin, inositol, isoleucyl-prolyl-proline (IPP), L-arginine, L-carnitine, L-cysteine, L-lysine, L-proline, L-taurine, lecithin, magnesium, manganese, molybdenum, niacin and niacinamide (vitamin B3), omega-3 fatty acids, pantothenic acid, pine bark extract, policosanols, pomegranate extract, potassium, protein, prune extract (fruit), quercetin, red wine extract, red wine (resveratol), rose hips, rutin, selenium, soy, turmeric, valyl-prolyl-proline (VPP), vitamin C (ascorbic acid), vitamin D, vitamin E (d-alpha-tocopherol), zinc, and mixtures thereof.

In one embodiment, the biologically active substance may include substances that support the subject's cardiovascular system by helping to reduce cholesterol. Substances that help to reduce cholesterol may be selected from, but not limited to, niacin and niacinamide (vitamin B3), beta-sitosterol, flaxseed, red yeast, sitostanol, alfalfa, artichoke, avocado, barley, calcium, English walnut, green tea, jiaogulan, macadamia nut, magnesium, olive, rice bran, safflower, sitostanol, soy, soybean oil, sweet orange, yogurt, amaranth, cod liver oil, garlic, guggul, inulin, lecithin, red clover, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

In one embodiment, the biologically active substance may include substances that support the subject's cardiovascular system by helping to reduce blood pressure. Substances that help to reduce blood pressure may be selected from, but not limited to, alpha-linolenic acid, calcium, cod liver oil, CoEnzyme Q10, fish oil, garlic, green tea, olive, Oolong tea, potassium, pycnogenol, stevia, sweet orange, vitamin C (ascorbic acid), wheat bran, eicosapentaenoic acid (EPA), gamma linolenic acid (GLA), vitamin E, casein peptides, cocoa, dimethylsulfoxide (DMSO), grape, hydroxymethylbutyrate (HMB), L-arginine, soy, tomato, yucca, guggul, alpha-linolenic acid, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

In one embodiment, the biologically active substance may include substances that support the subject's cardiovascular system by helping to reduce arteriosclerotic plaque. Substances that help to reduce arteriosclerotic plaque may be selected from, but not limited to, alpha-linolenic acid, black tea, fish oil, garlic, niacin and niacinamide (vitamin B3), vitamin C (ascorbic acid), vitamin E, Gotu kola, lycopene, mesoglycan, pomegranate, stevia, sweet orange, wheat bran, and mixtures thereof.

In one embodiment, the biologically active substance may include substances that support the subject's cardiovascular system by helping to lower LDL cholesterol and/or increase HDL cholesterol. Substances that help to lower LDL cholesterol and/or increase HDL cholesterol may be selected from, but not limited to, niacin, beta-sitosterol, flaxseed, red yeast, alfalfa, artichoke, avocado, hyperimmune egg, barley, calcium, English walnut, garlic, green tea, jiaogulan, macadamia nut, magnesium, olive, inositol nicotinate, policosanol, rice bran, safflower, sitostanol, soy, soybean oil, sweet orange, yogurt, amaranth, cod liver oil, guggul, inulin, lecithin, red clover, kefir, activated charcoal, aloe, bean pod, chitosan, docosahexaenoic acid (DHA), fenugreek, glucomannan, hydroxymethylbutyrate, pomegranate, pycnogenol, quercetin, royal jelly, sunflower oil, vitamin C, vitamin E, vitamin K, yucca, and mixtures thereof thereof.

In one embodiment, the biologically active substance may include substances that support the subject's cardiovascular system by helping improve cardiac output and/or increase the pumping strength of the heart. Substances that help to improve cardiac output and/or increase the pumping strength of the heart may be selected from, but not limited to, hawthorne berry, CoEnzyme Q10, taurine, carnithine, and mixtures thereof.

In another embodiment, the biologically active substance may include substances that support the subject's weight management control. Substances that support weight management control may be selected from, but not limited to, almond, Aloe vera, alpha lipoic acid, aminogen, ammonium glycyrrhizate, amylum fruit extract, astaxanthin, bean pod, benzyl alcohol, biotin, bitter orange, Piper nigrum L (black pepper) extract, Piper longum L (long pepper) extract, black tea extract, bladderwrack (kelp), blue-green algae, broccoli, butylene glycol, Indian Fig Opuntia cactus, caffeine, caralluma, carob, cassia seed extract, cayenne, calcium, calcium phosphate, cedarwood oil, cetyl alcohol, chitosan, HD, Cissus quadrangularis extract (stem & leaves), citrus lime oil, citrus orange oil, cocoa, CoEnzyme Q10, coix seed, cola nut, Coleus forsholii extract, cujquat (fruit), Combretum micranthum (leaf) extract, copper sulfate, cyclometicone, dandelion root, dehydroepiandrosterone (DHEA), 7-keto-DHEA, dill weed, diacylglycerol, dimethicone, disodium succinate, DL-phenylalanine, ephedra, flos citri auranti (blossoms), folic acid, Garcinia cambogia extract, geranium oil, ginger root, American root, fish oil, ginseng, panax extract, glyceryl stearate, grapefruit oil, green tea extract (leaf), guaiacwood oil, guarana extract, Gymnema sylvestre, Hoodia gordonia (stem), glucomannan, green tea, guggul, 5-hydroxy-tryptophan (5-HTP), inulin, kahkow fruit extract, lavendin oil, lecithin, hydroxylated, lemon grass, licorice, linoleic acid, L-camitine, L-glutamine, L-methionine, L-tyrosine, Lespedeza capitata extract, Litsea cubeba fruit oil, lotus leaf, magnolia, manganese, methyl paraben, milk protein isolate, mulberry (leaf), nettle leaf, niacin/niacinamide, Oolong tea extract (Camellia sinensis), pantothenic acid, papaya leaf, PEG-12/PEG-100, phaseolamin, phellodendron, picamilon HCl, pine leaf oil, potassium citrate, potassium iodide (iodine), potassium phosphate, Poria cocos (Fu Ling), propylparaben, pyruvate, quercetin, red clover blossom, Rhodiola rosea extract, rhubarb root (Da Huang), Rooibos tea extract (leaf & stem), rosemary leaf oil, sesame oil (Sesamum indicum), senna (leaf), Caralluma fimbriata, sodium benzoate, sodium caseinate, soy lecithin, soy protein isolate, Spanish sage oil, stevia leaf, sunflower oil, tangerine oil, tarragon extract, theobromine, threonine triethanolamine, tiratricol, Ulva lactuca extract, Arctostaphylos uva ursi leaf, vinpocetine, vitamin A, vitamin B1/B2, vitamin B6/B12, vitamin C (ascorbic acid), vitamin D3, vitamin E (d-alpha-tocopherol), water plantain rhizome (stem), whey powder, whey protein isolate, white willow bark extract, white pepper, Withania somnifera extract, Wu-Long tea, Yellowdock root, Yerba mate extract, zinc oxide, and mixtures thereof.

Without being bound by theory, it is believed the controlled release of the biologically active substance within a subject's digestive system is a phenomenon that is determined by the non-covalent interaction between the biologically active substance and the fiber side groups. This indicates that not all substances will exhibit the same magnitude of delayed release. Therefore, each individual biologically active substance may have its own optimal fiber content to maximize or customize its controlled release.

While specific embodiments of the present invention have been illustrated and described, numerous modifications come to mind without significantly departing from the spirit of the invention, and the scope of protection is only limited by the scope of the accompanying claims.

1. A method of providing controlled release of a biologically active substance within a subject's digestive system comprising:
obtaining an oral dosage unit comprising the biologically active substance combined with one or more soluble fibers, wherein the amount of the one or more soluble fibers in the oral dosage unit is greater than 40% by weight;
orally administering the oral dosage unit to the subject, wherein the one or more soluble fibers interact with the biologically active substance to provide controlled release of the biologically active substance within the subject's digestive system.

2. The method according to claim 1, wherein the one or more soluble fibers comprises a mixture of soluble fibers.

3. The method according to claim 1, wherein the one or more soluble fibers comprises a mixture of soluble fibers that includes soluble fibers selected from guar gum, gum Arabic, locust bean gum, pectin, oat fiber, beta glucan, psyllium, gum acacia, xanthane gum, innuline, fructo-oligosaccharides (FOS), carrageenan, and mixtures thereof.

4. The method according to claim 1, wherein the oral dosage unit comprises at least 1 g of the soluble fiber.

5. The method according to claim 1, wherein the oral dosage unit comprises from about 1 to 15 g of the soluble fiber.

6. The method according to claim 1, wherein the oral dosage unit comprises from about 2 to 10 g of the soluble fiber.

7. The method according to claim 1, wherein the oral dosage unit comprises from about 3 to 5 g of the soluble fiber.

8. The method according to claim 1, wherein the amount of the one or more soluble fibers in the oral dosage unit is from about 40% to about 95% by weight of the oral dosage unit.

9. The method according to claim 1, wherein the amount of the one or more soluble fibers in the oral dosage unit is from about 45% to about 90% by weight of the oral dosage unit.

10. The method according to claim 1, wherein the amount of the one or more soluble fibers in the oral dosage unit is from about 45% to about 75% by weight of the oral dosage unit.

11. The method according to claim 1, wherein the amount of the one or more soluble fibers in the oral dosage unit is from about 45% to about 60% by weight of the oral dosage unit.

12. The method according to claim 1, wherein the amount of the one or more soluble fibers in the oral dosage unit is from about 45% to about 55% by weight of the oral dosage unit.

13. The method according to claim 1, wherein the oral dosage unit comprises one or more insoluble fibers.

14. The method according to claim 1, wherein the oral dosage unit further comprises an orally-ingestible pharmacologically-acceptable mineral salt capable of dissolution in the digestive system to release a gas.

15. The method according to claim 14, wherein the mineral salt comprises a mineral carbonate compound, a mineral hydrogen carbonate compound, a mineral dihydrogen carbonate compound, or mixtures thereof.

16. The method according to claim 1, wherein the oral dosage unit is administered to the subject substantially simultaneously with a meal.

17. The method according to claim 1, wherein the biologically active substance comprises nutritional vitamins, minerals, and phytonutrients.

18. The method according to claim 17, wherein the nutritional vitamins, minerals and phytonutrients comprise fruit and vegetable derived vitamins, minerals, phytonutrients, or mixtures thereof.

19. The method according to claim 18, wherein the fruit and vegetable phytonutrients are selected from, acai, alfalfa, apple, artichoke, apricot, asparagus, avocado, barley grass, bilberry, beans, bittermelon, beet, blackberry, broccoli, black current, Brussels sprouts, blueberry, cabbage, cantaloupe, cassava, carrot, cherry, cauliflower, coconut, celery, coriander, cranberry, chlorella, gauvas, corn, grape, cucumber, garlic, grapefruit, horseradish, hops, kale, kava, kamut, kiwi, lima beans, lemon, oat grass, mangos, olive, orange, parsley, papaya, peach, peach, peas, pear, pepper, pineapple, potato, plum, pumpkin, pomegranate, rice, raspberries, spinach, strawberry, spirulina, tangerines, squash, tomato, sweet potatoes, wheat germ, wheat grass, white kidney beans, and mixtures thereof.

20. The method according to claim 17, wherein the vitamins and phytonutrients comprise antioxidants.

21. The method according to claim 20, wherein the antioxidants are selected from lycopene, anthocyanosides, alfalfa, chlorophyll, beta-carotene, alpha-carotene, lutein, zeaxanthin, canthaxanthin, astaxanthin, tocopherol, epigallocatechin gallate (EGCG), acetylcysteine, alpha lipoic acid, bilberry, burdock, carnosine, catalase, conjugated linoleic acid (CLA), CoEnzyme Q10, cryptoxanthin, curcumin, daidzein, dehydroepiandrosterone (DHEA), dimethylaminoethanol (DMAE), garlic, genistein, germanium, Ginkgo biloba, glutamine, glutathione, grape seed extract, green tea, lutein, lycopene, manganese, melatonin, methionine, para-aminobenzoic acid (PABA), pycnogenol, quercetin, resveratrol, selenium, superoxide dismutase, taurine, vitamin C (ascorbic acid), vitamin E, zeaxanthin, zinc, and mixtures thereof.

22. The method according to claim 17, wherein the phytonutrients comprise antioxidant plant enzymes.

23. The method according to claim 22, wherein the antioxidant plant enzymes are selected from catalase, glucose oxidase, peroxidase, superoxide dismutase, glutathione peroxidase, and mixtures thereof.

24. The method according to claim 17, wherein the nutritional vitamins and minerals comprise antioxidant vegetable concentrates.

25. The method according to claim 1, wherein the biologically active substance comprises substances that support the subject's immune system.

26. The method according to claim 25, wherein the substances that support the immune system are selected from, alfalfa leaf, alpha lipoic acid, allium cepa, aloe vera, antioxidant plant enzymes, apricot extract, nectarine extract, arabinogalactan, Arnica montana, arsenicum album, bee pollen, benzenum, belladonna, beta carotene, biotin, Piper nigrum L (black pepper) extract, Piper longum L (long pepper) extract, bladderwrack (kelp) extract, boron, colostrum, burdock root, cadmium sulphuricum, calcium ascorbate, calcium citrate, capsicum, carotenoids, carrot, cat's claw, cayenne, chlorum, choline bitartrate, polynicotinate, citrus bioflavonoids (lemon), cruciferous vegetable concentrate, cupric oxide (copper), cuprum metallicum, copper glycinate, dandelion root, Drosera rotundifolia, echinacea, elderberry, ferrum phosphoricum, folic acid (folate), fructose, garlic, ginger root, golden seal root, grape seed extract, grape wine concentrate, green tea extract (leaf), guarana extract, xanthan gum, hops strobile, inositol, iron rice chelate, lactoferrin, lutein, lycopene, magnesium, manganese rice chelate, marigold flower extract, milk thistle herb, mixed berry anthocyanosides, molybdenum, Morinda citrifolia extract, mulberry, n-acetyl cysteine, nettle leaf, niacin/niacinamide, nux vomica, oxo vanadium bis glycinato, pantothenic acid, para amino benzoic acid (PABA), perilla seed extract, pine bark extract, plumbum metallicum, pomegranate extract, potassium, potassium iodide (iodine), prune extract (fruit), quercetin, red clover blossom, red wine extract, rhubarb root, rose hips, rutin, Schisandra chinensis fruit extract, scullcap herb, selenium, sheep sorrel herb, slippery elm bark, soy, tabacum, turmeric, valerian root, vitamin A palmitate, vitamin B1/B2, vitamin B6/B12, vitamin C (ascorbic acid), vitamin D (2&3), vitamin E (d-alpha-tocopherol), vitamin K (phytonadione), watercress leaf, CoEnzyme Q10, glutamine, hydroxymethylbutyrate (HMB), L-arginine, lentinan, red yeast, S-adenosyl- L-methionine (SAMe), sangre de grado (dragon's blood), whey protein, medium chain triglycerides (MCT), St. John's Wort, boxwood, dehydroepiandrosterone (DHEA), riboflavin (vitamin B2), Uña de Gato extract, zinc, and mixtures thereof.

27. The method according to claim 1, wherein the biologically active substance comprises substances that support the subject's cardiovascular system.

28. The method according to claim 27, wherein the substances that support the subject's cardiovascular system are selected from acerola, apricot extract, nectarine extract, B-Vitamins (1, 2, 3, 6, 12), beta carotene, biotin, black current seed oil, gamma-linolenic acid (GLA), borage oil, calcium phosphate, carnosine, carotenoids, choline bitartrate, chondroitin sulfate, coconut oil, cognis phytosterols, copper (cupric oxide), CoEnzyme Q10, D-ribose, evening primrose seed oil, fish oil, eicosapentaenoic acid (EPA), docosahexaenoic acid (DHA), flax seed oil, folic acid (folate), garlic, grape seed extract, grape juice extract, grape skin extract, green tea extract, glutathione (GSH), hawthorne berry extract, hesperidin, inositol, isoleucyl-prolyl-proline (IPP), L-arginine, L-carnitine, L-cysteine, L-lysine, L-proline, L-taurine, lecithin, magnesium, manganese, molybdenum, niacin and niacinamide (vitamin B3), omega-3 fatty acids, pantothenic acid, pine bark extract, policosanols, pomegranate extract, potassium, protein, prune extract (fruit), quercetin, red wine extract, red wine (resveratol), rose hips, rutin, selenium, soy, turmeric, valyl-prolyl-proline (VPP), vitamin C (ascorbic acid), vitamin D, vitamin E (d-alpha-tocopherol), zinc, and mixtures thereof.

29. The method according to claim 27, wherein the substances that support the subject's cardiovascular system are selected from niacin and niacinamide (vitamin B3), beta-sitosterol, flaxseed, red yeast, sitostanol, alfalfa, artichoke, avocado, barley, calcium, English walnut, green tea, jiaogulan, macadamia nut, magnesium, olive, rice bran, safflower, sitostanol, soy, soybean oil, sweet orange, yogurt, amaranth, cod liver oil, garlic, guggul, inulin, lecithin, red clover, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

30. The method according to claim 27, wherein the substances that support the subject's cardiovascular system are selected from alpha-linolenic acid, calcium, cod liver oil, CoEnzyme Q10, fish oil, garlic, green tea, olive, Oolong tea, potassium, pycnogenol, stevia, sweet orange, vitamin C (ascorbic acid), wheat bran, eicosapentaenoic acid (EPA), gamma linolenic acid (GLA), vitamin E, casein peptides, cocoa, dimethylsulfoxide (DMSO), grape, hydroxymethylbutyrate (HMB), L-arginine, soy, tomato, yucca, guggul, alpha-linolenic acid, kefir, activated charcoal, aloe, bean pod, chitosan, cocoa, docosahexaenoic acid (DHA), fenugreek, flaxseed oil, glucomannan, hydroxymethylbutyrate (HMB), hyperimmune egg, inositol nicotinate, Job's Tears (Coix lacryma-jobi), policosanol, pomegranate, pycnogenol, quercetin, royal jelly, sunflower oil, vitamin C (ascorbic acid), vitamin E, vitamin K, yucca, and mixtures thereof.

31. The method according to claim 27, wherein the substances that support the subject's cardiovascular system are selected from alpha-linolenic acid, black tea, fish oil, garlic, niacin and niacinamide (vitamin B3), vitamin C (ascorbic acid), vitamin E, Gotu kola, lycopene, mesoglycan, pomegranate, stevia, sweet orange, wheat bran, and mixtures thereof.

32. The method according to claim 1, wherein the biologically active substance comprises substances that support the subject's weight management control.

33. The method according to claim 32, wherein the substances that support the that support the subject's weight management control are selected from almond, Aloe vera, alpha lipoic acid, aminogen, ammonium glycyrrhizate, amylum fruit extract, astaxanthin, bean pod, benzyl alcohol, biotin, bitter orange, Piper nigrum L (black pepper) extract, Piper longum L (long pepper) extract, black tea extract, bladderwrack (kelp), blue-green algae, broccoli, butylene glycol, Indian Fig Opuntia cactus, caffeine, caralluma, carob, cassia seed extract, cayenne, calcium phosphate, cedarwood oil, cetyl alcohol, chitosan, Cissus quadrangularis extract (stem & leaves), citrus lime oil, citrus orange oil, cocoa, CoEnzyme Q10, coix seed, cola nut, Coleus forsholii extract, cujquat (fruit), Combretum micranthum (leaf) extract, copper sulfate, cyclometicone, dandelion root, dehydroepiandrosterone (DHEA), 7-keto-DHEA, dill weed, diacylglycerol, dimethicone, disodium succinate, DL-phenylalanine, ephedra, flos citri auranti (blossoms), folic acid, Garcinia cambogia extract, geranium oil, ginger root, American root, fish oil, ginseng, panax extract, glyceryl stearate, grapefruit oil, green tea extract (leaf), guaiacwood oil, guarana extract, Gymnema sylvestre, Hoodia gordonia (stem), glucomannan, green tea, guggul, 5-hydroxytryptophan (5-HTP), inulin, kahkow fruit extract, lavendin oil, lecithin, hydroxylated, lemon grass, licorice, linoleic acid, L-carnitine, L-glutamine, L-methionine, L-tyrosine, Lespedeza capitata extract, Litsea cubeba fruit oil, lotus leaf, magnolia, manganese, methyl paraben, milk protein isolate, mulberry (leaf), nettle leaf, niacin/niacinamide, Oolong tea extract (Camellia sinensis), pantothenic acid, papaya leaf, PEG-12/PEG-100, phaseolamin, phellodendron, picamilon HCl, pine leaf oil, potassium citrate, potassium iodide (iodine), potassium phosphate, Poria cocos (Fu Ling), propylparaben, pyruvate, quercetin, red clover blossom, Rhodiola rosea extract, rhubarb root (Da Huang), Rooibos tea extract (leaf & stem), rosemary leaf oil, sesame oil (Sesamum indicum), senna (leaf), Caralluma fimbriata, sodium benzoate, sodium caseinate, soy lecithin, soy protein isolate, Spanish sage oil, stevia leaf, sunflower oil, tangerine oil, tarragon extract, theobromine, threonine triethanolamine, tiratricol, Ulva lactuca extract, Arctostaphylos uva ursi leaf, vinpocetine, vitamin A, vitamin B1/B2, vitamin B6/B12, vitamin C (ascorbic acid), vitamin D3, vitamin E (d-alpha-tocopherol), water plantain rhizome (stem), whey powder, whey protein isolate, white willow bark extract, white pepper, Withania somnifera extract, Wu-Long tea, Yellowdock root, Yerba mate extract, zinc oxide, and mixtures thereof.
